(19) United States
(12) Patent Application Publication
(10) Pub. No.: US 2017/0128467 A1
(43) Pub. Date: May 11, 2017

(54) ANTIMICROBIAL PHARMACEUTICAL COMPOSITIONS WITH MULTIPLE DRUG RESISTANCE INHIBITING PROPERTIES

(71) Applicant: Lawrence T. Friehoff, River Vale, NJ (US)

(72) Inventors: Lawrence T Friedhoff, River Vale, NJ (US); Maxim Vladimirovich Skulachev, Moscow (RU); Fedor Fedorovich Severin, Moscow (RU); Dmitry Alexeevich Knorre, Moscow (RU); Julia Evgenyevna Karavaeva, Protvino (RU); Pavel Alexandrovich Nazarov, Moscow (RU); Yuri Nikolaevich Antonenko, Moscow (RU)

(21) Appl. No.: 15/315,246

(22) PCT Filed: May 29, 2015
(86) PCT No.: PCT/US15/33242

§ 371 Date: Nov. 30, 2016

Related U.S. Application Data
(60) Provisional application No. 62/005,128, filed on May 30, 2014.

(51) Int. Cl. A61K 31/66; A61K 45/06

(52) U.S. Cl. CPC A61K 31/66; A61K 45/06

(57) ABSTRACT

The invention provides a method for treating a microbial infection in an infected subject comprising administering to the subject an antimicrobial composition comprising a therapeutically effective amount of one or more lipophilic cation, and, optionally, another one or more antimicrobial compound or antimicrobial agent. The invention further provides a pharmaceutical composition with antimicrobial properties comprising a therapeutically effective amount of one or more lipophilic cation and another one or more antimicrobial compound or antimicrobial agent.

### BACKGROUND OF THE INVENTION

Field of the Invention

The invention relates to pharmaceutical compositions having antimicrobial properties. The invention further relates to such pharmaceutical compositions having multiple drug resistance-inhibiting properties.

Summary of the Related Art

Antibiotics represent one of medical science's greatest advancements in history. Unfortunately, the overuse of certain antibiotics in medicine and in agriculture have lead to the emergence of pathological microbes that are resistant to many of the most commonly used antibiotics, necessitating the need for structurally distinct antibiotics to which such microbes have not been exposed. One form such antibiotic resistance is due to some antibiotics being able to activate multiple drug resistance (MDR) systems in different cells including fungi and bacteria. As a result these MDR systems pump out the antibiotics, thereby reducing their concentration in the target cells and significantly decreasing the antimicrobial effect of the antibiotics. For example, in many cases pathogenic fungi possess a robust MDR system (see references 1 and 2). Development of safe, convenient and universal methods of inhibition of microbial MDR pumps is an unsolved problem yet. An effective antibiotic that is also an MDR inhibitor is expected to display antibacterial or anti fungal properties that are less susceptible to MDR-mediated resistance because it increases the concentration of the antibiotic in the target cell by shifting the balance of influx/efflux of these antibiotics. In turn this shift of balance and increase of concentration of antibiotics should decrease the viability of the target cell.

Lipophilic cations have been extensively studied in during last 10-15 years as mitochondrially targeted pharmaceuticals. The major effect of such pharmaceuticals is the protection of target cells from from oxidative stress and other stress factors (see, for example Lukashev et al, 2014). These protective properties have been shown for many compounds of the SkQ family (see reference 3, WO2011059355) as well as for lipophilic cations lacking an antioxidant moiety, for example C12TPP, C12R19 and others (see WO2011162633 and references 15, 16). These mitochondrially targeted (i.e. lipophilic and at the same time positively charged) compounds can be accumulated inside bacteria and fungi because of electric potential on the outer membrane of the cells of these organisms. Thus it could be expected that treatment of bacteria of fungi with these compounds can protect these organisms in stress conditions and increase their viability. Therefore it could be considered that these lipophilic cation compounds should not be used as a treatment of bacterial of fungi infection. There is, therefore, a need for antibiotics that also have MDR-inhibiting properties.

### BRIEF SUMMARY OF THE INVENTION

The invention provides pharmaceutical formulations of antibiotics that also have MDR-inhibiting properties, and methods for their use as antibacterial and antifungicidal agents. The present inventors have surprisingly discovered that lipophilic cationic compounds previously shown to have protective properties for cells actually have antibiotic properties for bacteria and fungi, while simultaneously having MDR-inhibiting properties.

Generally such lipophilic cation compounds have the structural formula 1:

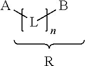

wherein A is hydrogen atom (H) or an antioxidant moiety having the following structure:

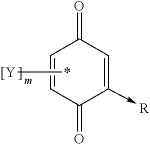

and/or reduced (i.e. quinole) form thereof
wherein m is an integer from 0 to 3; each Y is methyl;
L is a linker group which is straight or branched hydrocarbon chain which can be optionally substituted by one or more substituents and optionally contains one or more double or triple bonds; and
B is a lipophilic cation. Such compounds are charged (cations), thus they are present in the form of salt with any pharmacologically acceptable anion (counterion).

The methods according to the invention comprise administering to a subject having a bacterial or fungal infection a pharmaceutically effective amount of one or more lipophilic cationic compound according to the invention, optionally with another one or more antimicrobial compound or one or more antimicrobial agent.

### BRIEF DESCRIPTION OF THE DRAWINGS

FIG. 1 shows that expression levels of Pdr5 affect resistances to C₁₂TPP. FIG. 1A show the chemical structure of dodecyltriphenylphosphonium C₁₂TPP and its plastoquinone derivative (SkQ1) used in this study. FIG. 1B shows the results of glucose-grown cells treated with indicated concentration of C₁₂TPP or SkQ1. FIG. 1C shows the results of galactose-grown cells treated with indicated concentration of C₁₂TPP or SkQ1. *P<0.05 compared to untreated WT according to Wilcoxon signed-ranked unpaired test.

FIG. 2 shows that C₁₂TPP or energy deprivation enhances Rhodamine 6G accumulation in yeast cells. FIG. 2A shows that R6G staining of yeast cells is enhanced by low concentrations of C₁₂TPP but not FCCP. Exponentially grown yeast cells were stained with 500 nM R6G in the presence of indicated concentrations of C₁₂TPP or FCCP. FIG. 2B shows that addition of glucose results in a decrease of total fluorescence. Weak signals are observed from R6G retained in mitochondria (enhanced contrast). (Representative photographs; bars, 5 μm).

FIG. 3 shows that C₁₂TPP and SkQ1 prevent R6G efflux from yeast cells. FIG. 3A shows indirect measurements of glucose-induced R6G efflux from yeast cells. FIG. 3B shows that glucose-induced R6G efflux is negligible from AD1-8 (MDR-negative) cells. FIGS. 3C and D show that FCCP (2 μM), C₁₂TPP (2 μM) or SkQ1 (2 μM) inhibits glucose-induced R6G efflux from yeast cells. FIG. 3E shows quantification of the results. The ordinate corresponds to the ratio of slopes in a and b. FIG. 3F shows that FCCP (2 μM), C₁₂TPP (2 μM) or SkQ1 (2 μM) inhibits glucose-induced R6G efflux from yeast cells pretreated with 10 mM NaN₃. *P<0.05, **P<0.01 compared with untreated WT according to Wilcoxon signed-ranked unpaired test.

FIG. 4 shows that C₁₂TPP enhances the effects of Pdr5 substrates cycloheximide D and clotrimazole. FIG. 4A shows duplication times of yeast cells grown in the presence of ethanol (mock control), C₁₂TPP (1 μM), Cycloheximide D (ChD, 0.05 μM), or both chemicals. FIG. 4B shows that C₁₂TPP (1 μM) increases the inhibitory effect of Clotrimazole (CltrA, 60 μM). *P<0.05 compared with untreated WT according to Wilcoxon signed-ranked paired test.

FIG. 5 shows that overexpression of PDR5 gene increases active efflux of C12R1 from yeast cells. To measure the relative rate of C12R1 efflux, the fluorometric assay as in Knorre et al. 2014 (http://dx.doi.org/10.1016/j.bbrc.2014.07.017) was used, except that, instead R6G energy deprived cells were stained with 10 μM C12R1. The control laboratory strain W303-1A (WT) and the strain PGAL-PDR5 were grown on galactose containing media (conditions of PGAL overexpression). To repress PDR5 gene cells were grown on rich medium containing glucose (conditions of PGAL repression).

FIG. 6 shows that C12TPP prevents C12R1 efflux from yeast cells. The rate of C12R1 efflux (the slope of fluorescence increase on FIG. 1) was quantified in cells grown either on YPD (yeast peptone dextrose; conditions of PGAL repression), or YPGAL (yeast peptone galactose; conditions of PGAL overexpression). The measurements was conducted with supplementation of 10 μM C12TPP where indicated.

FIG. 7 shows that C₁₂R1 facilitates antifungal effect of clotrimazole (Cltr) and benzalkonium chloride (BnzCl). Yeast cells were grown in liquid YPD medium with indicated chemicals for 2 hours, then cells were plated on solid YPD and the number of emerged colony forming units (CFU) were calculated after 2 days. 100% corresponds to CFU in yeast suspension before addition of chemicals, control bar corresponds to the number of CFU after 2 hours of incubation without chemicals.

FIG. 8 shows growth curves of B. subtilis and E. coli in the presence and absence of SkQ1, as measured by absorption at 600 nanometers. Inhibitors added at zero timepoint in the range of concentration of 1-100 μM.

FIG. 9 shows effects of different CₙTPP concentrations on growth of B. subtilis and E. coli. Zero point indicates MIC for CₙTPP.

FIG. 10 shows the effects of different compounds of formula 1 on growth of bacteria of B. subtilis in the LB media measured by absorption at 600 nanometers. Inhibitors were added at zero timepoint in the range of concentration of 1-100 μM.

FIG. 11 shows antibacterial activity of SkQ1 on growth of bacteria ΔTo1C and ΔAcrA and ΔAcrB. Inhibitors were added at zero timepoint.

FIG. 12 shows antibacterial activity of CₙTPP on growth of bacteria ΔTo1C strain. Inhibitors added at zero timepoint.

FIG. 13 shows accumulation of ethidium bromide in presence of SkQ1. For emulation of cell membrane leakage, bacterial cells were resuspended in deionized water. Ethidium bromide was added (20 μg/ml) and the change of fluorescence intensity was recorded on Fluorat-02-Panorama. For detecting of the accumulation of ethidium bromide, SkQ1 was added to a concentration of 50 μM.

### DETAILED DESCRIPTION OF THE PREFERRED EMBODIMENTS

The invention relates to pharmaceutical compositions having antimicrobial properties and methods of their use to treat microbial infections. The invention further relates to such pharmaceutical compositions having multiple drug resistance-inhibiting properties. The invention provides pharmaceutical formulations of antibiotics that also have MDR-inhibiting properties, and methods for their use as antibacterial and antifungal agents. The present inventors have surprisingly discovered that lipophilic cationic compounds previously shown to have protective properties for cells actually have antibiotic properties for bacteria and fungi, while simultaneously having MDR-inhibiting properties.

Generally such lipophilic cation compounds have the structural formula 1:

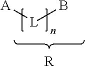

wherein A is hydrogen atom (H) or an antioxidant moiety having the following structure:

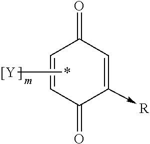

and/or reduced (i.e. quinole) form thereof
wherein m is an integer from 0 to 3; each Y is methyl;
L is a linker group which is straight or branched hydrocarbon chain which can be optionally substituted by one or more substituents and optionally contains one or more double or triple bonds; and
B is a lipophilic cation. Such compounds are charged (cations), thus they are present in the form of salt with any pharmacologically acceptable anion (counterion).

The methods according to the invention comprise administering to a subject having a bacterial or fungal infection a pharmaceutically effective amount of one or more lipophilic cationic compound according to the invention, optionally with another one or more antimicrobial compound or one or more antimicrobial agent.

Compounds useful in the invention include, without limitation, the following exemplary compounds: SkQ1, SkQ3, SkQ4, SkQ5, SkQR1, SkQRB, SkQB1, SkQBP1, SkQT, SkQThy, SkQB, CₙTPP (wherein n is from 5 to 12), CₙR1 (wherein n is from 4 to 12), CₙBerb (wherein n is from 4 to 12), and CₙPalm (wherein n is from 4 to 12). Structural formulae of representative compounds are presented below.

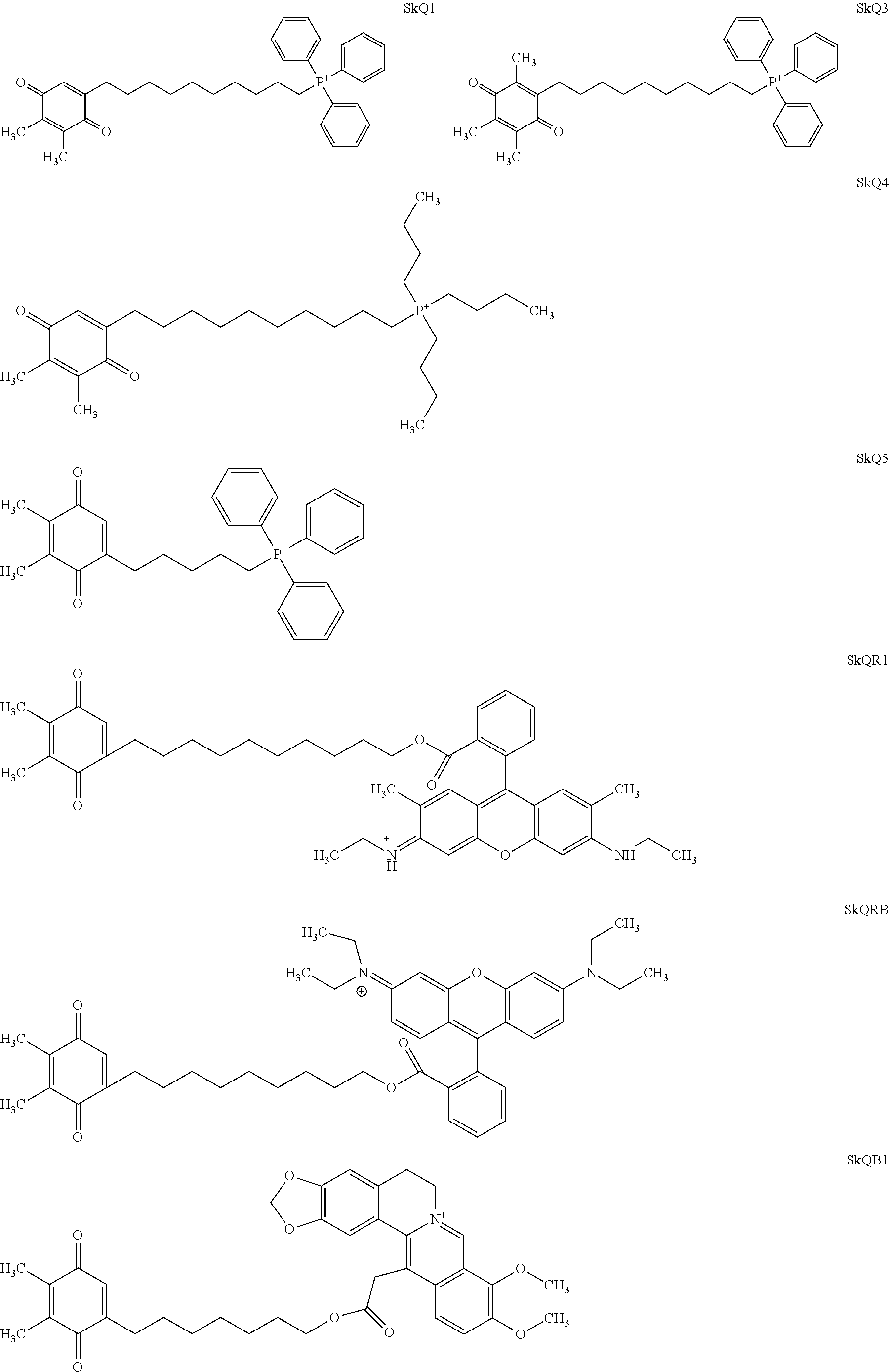

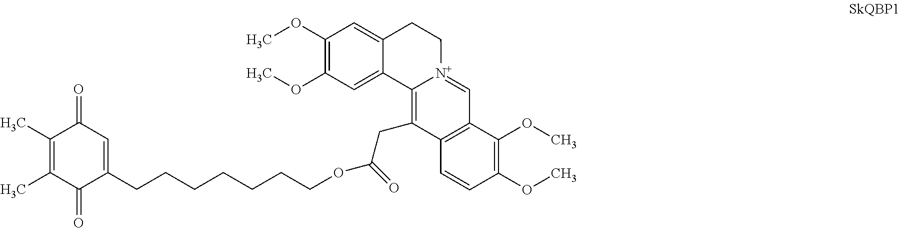

SkQT (mixture of isomers that differ by the position of decyl linker, possible positions are indicated with arrows)

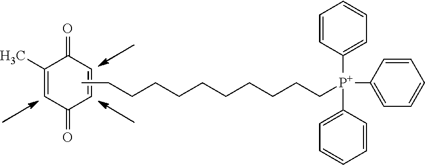

Among these SkQT isomers there is SkQT-para:

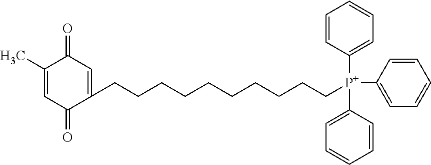 [SMILES: CC1=CC(=O)C(CCCCCCCCCC[P+](c2ccccc2)(c2ccccc2)c2ccccc2)=CC1=O]

SkQThy (mixture of isomers that differ by the position of decyl linker, possible positions are indicated with arrows)

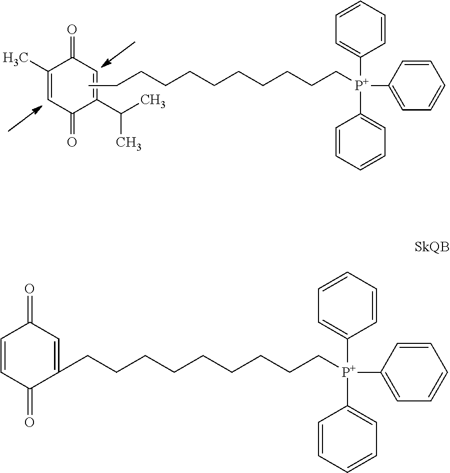

Compounds useful in the invention also include reduced (quinole) forms of above mentioned compounds. In these reduced compounds the quinone moiety:

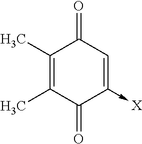

is reduced, i.e. replaced with quinole moiety:

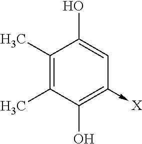

For example in case of SkQ1 the reduced form SkQ1 H₂ has the following structure:

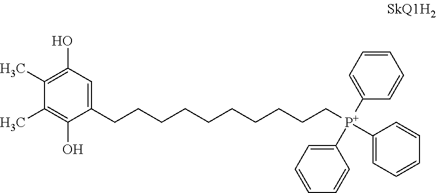 [SMILES: Cc1c(O)cc(CCCCCCCCCC[P+](c2ccccc2)(c2ccccc2)c2ccccc2)c(O)c1C]

Also as an example, in case of SkQ3 the reduced form SkQ3H₂ has the following structure:

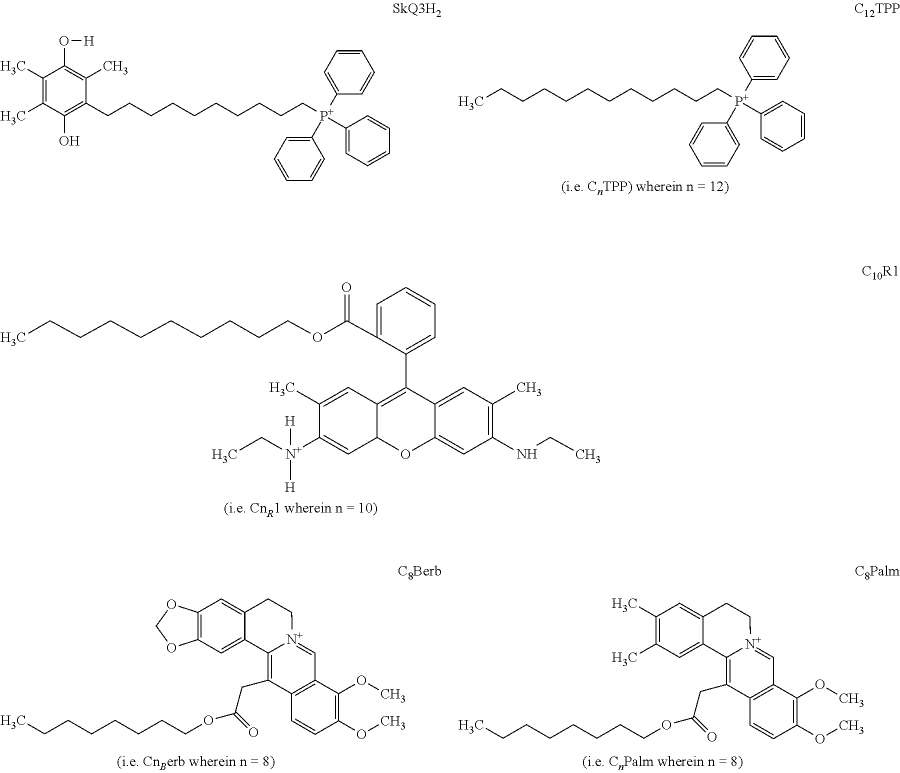

An important feature of the compounds of general formula 1 as anti-microbial agents is the positive charge of these compounds. The precise structure of the positively charged moiety is not particularly important, as long as the moiety contains sufficient hydrophobic character to spread out the positive charge, such that the overall compound is lipophilic. Once such compounds are expelled from a microbe cell by MDR or other protection system, the compounds are able to penetrate back into the cell due to electric charge on the outer membrane of the microbe (with negative charge inside the cell). This feature makes compounds of general formula 1 recyclable anti-microbial agents.

In our experiments we have unexpectedly found that different compounds of the general formula 1 provide antimicrobial (i.e. anti-bacteria or anti-fungal) effect. Partially, this effect can be explained by the ability of these compounds to suppress MDR systems in bacteria or fungi. These findings provide a new area of application for the compounds of general formula 1. This area of application is the treatment of different infectious diseases caused by fungi, bacteria, or some viruses. As demonstrated by our experimental examples, antimicrobial properties of compounds of general formula 1 allow this treatment to be performed by a compound of general formula 1 alone, i.e. in monotherapy. Another set of our experimental examples demonstrated that compounds of general formula 1 are able to suppres MDR system in bacteria or fungi. Thus another aspect of our invention is application of compounds of general formula 1 in combination with another antimicrobial agent. This agent can be an antibiotic (anti-bacterial agent), an antimycotic (anti-fungi agent) or an antiseptic.

Previous studies of lipophilic cations have demonstrated that these compounds have anti-inflammatory properties (WO2012167236, WO2014116591, WO2011162633, Zinovkin R. A. et al, Aging (Albany, N.Y.). 2014 ;6(8):661-74). Thus another aspect of this invention is the use of the benefit of compounds of general formula 1 as an agent providing both anti-microbial and anti-inflammatory effects. Yet another aspect of the invention is a method of treatment of a patient who would benefit from simultaneous suppression of infection and inflammation.

Within the scope of this invention, the treatment of infection can be systemic or local. Systemic treatments include, without limitation, oral administration, intravenous injection, nasal administration, rectal administration of a compound of general formula 1 to the patient. Local treatments include, without limitation, administration in a form of eye-drops, gels, ointments, lotions, sprays, bandages.

Experimental examples and examples of compositions are presented below solely to illustrate the applicability of invention and cannot be regarded as examples limiting the scope of claims of the present invention. The following abbreviations are used in the examples:

SkQ1—plastoquinonildecyltriphenylphosphonium.

C₁₂TPP—dodecyltriphenylphosphonium

R6G—rhodamine 6G

MDR—multiple drug resistance

FCCP—cyanide p-trifluoro-methoxyphenyl hydrazone

ABC—ATP binding cassette

MIC—minimal inhibitory concentration.

### Examples of Compositions

In general the compositions provided by the invention includes effective amount of membrane penetrating cation alone and/or with another anti-bacterial or ant-fungal agent. Below are several non-limiting examples of possible compositions:

(1) Compositions comprising an effective amount of one or more membrane penetrating cation according to the invention and an effective amount of one or more ergosterol synthesis inhibitor, including, without limitation, one or more of Ketoconazole or Itraconazole, Fluconazole, Voriconazole, Posoconazole, Ravuconazole, Bifonazole, Butoconazole, Clomidazole, Clotrimazole, Croconazole, Econazole, Fenticonazole, Isoconazole, Miconazole, Neticonazole, Omoconazole, Oxiconazole, Sertaconazole, Sulconazole, Tioconazole, Terconazole, and Hexaconazole.

(2) Compositions comprising an effective amount of one or more membrane penetrating cation according to the invention and an effective amount of one or more ergosterol membrane disrupter, including, without limitation, one or more of amphotericin B, Hamycin, Natamycin, and Nystatin.

(3) Compositions comprising an effective amount of one or more membrane penetrating cation according to the invention and effective amount of one or more squalene epoxidase inhibitor, including, without limitation, one or more of morpholines, Amorolfine, Terbinafine, and Naftifine.

(4) Compositions comprising an effective amount of one or more membrane penetrating cation according to the invention and an effective amount of one or more β-glucan synthase inhibitor, including, without limitation, one or more of Anidulafungin, Caspofungin, and Micafungin.

(5) Compositions comprising an effective amount of one or more membrane penetrating cation according to the invention and an effective amount of one or more chitin synthesis inhibitor, including, without limitation, nikkomycin and polyoxins (6) Compositions comprising an effective amount of one or more membrane penetrating cation according to the invention and an effective amount of one or more fungi-specific protein synthesis inhibitor, including, without limitation, sordarins.

(7) Compositions comprising an effective amount of one or more membrane penetrating cation according to the invention and an effective amount of one or more thymidylate synthase inhibitor, including, without limitation, Flucytosine.

(8) Compositions comprising an effective amount of one or more membrane penetrating cation according to the invention and an effective amount of one or more fungi specific mitotic inhibitor, including, without limitation, Griseofulvin.

(9) Compositions comprising an effective amount of one or more membrane penetrating cation according to the invention and an effective amount of one or more antimicotic, including, without limitation, one or more of Bromochlorosalicylanilide, Methylrosaniline, Tribromometacresol, Undecylenic acid, Polynoxylin, Chlorophetanol, Chlorphenesin, Ticlatone, Sulbentine, Ethylparaben, Haloprogin, Salicylic acid, Selenium sulfide, Ciclopirox, Amorolfine, Dimazole, Tolnaftate, Tolciclate, and Taurolidine.

### EXPERIMENTAL EXAMPLES

### Experimental Example 1

### Anti-Fungi Effect of Compounds of General Formula 1

In this experiment we used yeast Saccharomyces cerevisiae as a model fungi. We have found that ABC-transporter Pdr5p protects the cells from the anti-fungi effects of C₁₂TPP, a compound of general formula 1. In agreement with this, we show that C₁₂TPP augments the toxic effects of Pdr5p substrates cycloheximide D and clotrimazole and also inhibits rhodamine 6G efflux. This demonstrates that C₁₂TPP is an effective competitive inhibitor of MDR. An important feature of C₁₂TPP and other penetrating lipophilic cations is their ability to reenter cell due to electric charge on the outer membrane of the cell. Thus, C₁₂TPP and other penetrating lipophilic cations act as recyclable MDR inhibitors demonstrating high efficiency.

Strains and Growth Conditions

In this example, we used W303-1A S. cerevisiae strains and its derivatives: AD1-8 with deletions of eight MDR genes [W303-1A, yor1::hisG, snq2::hisG, pdr5:shisG, pdr10::hisG, pdr11::hisG, ycf1::hisG, pdr3::hisG, pdr15::hisG][4]□, P_{GAL}-PDR5 [W303-1A HIS3::P_{GAL}-PDR5], P_{GAL}-SNQ2 [W303-1A HIS3::P_{GAL}-SNQ2], and P_{GAL}-M1H1 [W303-1AHIS3::P_{GAL}-YOR1](this study). Cells were grown in YPD medium (2% glucose, 1% bactopeptone, 1% yeast extract), or in YPGal (2% galactose, 1% bactopepton, 1% yeast extract). For genetic screening and maintenance of strains with conditionally expressed genes synthetic drop-out media YNB-Leu or YNB-His were used according to Sherman 2000 [5]. The growth rates were measured by increase of light scattering (λ=550 nm) in liquid yeast culture.

Microscopy

Cells stained with R6G were visualized with an upright Olympus BX2 microscope and U-MNG2 filter set (excitation 530-550 nm, 570 nm beamsplitter filter, emission >590 nm).

Genetic Screening.

Yeast mutants of W303 strain of S. cerevisiae carrying multicopy plasmid YEp13 with inserts of 8-10 Kb at BamHI restriction site have been constructed by transformation. Three cycles of enrichment of mutant collection for C₁₂TPP resistant strains were performed. During each cycle the mutants at logarithmic stage of growth on YNB-LEU media were treated with 18 μM C₁₂TPP for 3 hours, then washed, diluted and grown overnight on fresh solid YNB-LEU. After the third cycle cells were transferred on solid YNB-LEU media. C₁₂TPP resistance of separated colonies was compared with a wild type. To identify the genes carried by the multicopy plasmid, the genomic DNAs of the selected strains were transformed in E. coli. Loci of insertion were determined by sequencing the selected YEp13-insertion plasmids with primers YEp13-DIR 5′-cgctatatgcgttgatgc YEp13-REV 5′-cctgccaccatacccacg.

Rhodamine 6G Efflux

To measure the relative rate of rhodamine 6G efflux we used fluorometric assay implemented by Kolaczkowsky et al. [6], with few modifications. Cells were grown overnight in 40 ml in liquid YPD to density of 0.5-1*10⁷ cells/ml, washed twice with cold sterile water and resuspended in 10 ml phosphate buffer saline supplemented with 5 mM 2-deoxyglucose and 2.5 mM 2,4-dinitrophenol. Cell suspension was incubated for 45 minutes on a rotatory shaker, then the inhibitors were removed by two cycles of centrifugation/resuspension in cold water. The energy deprived cells were resuspended in 10 ml of PBS and then stained with R6G (10 μM) for 30 minutes. Then cell suspension was pelleted, resuspended in equal volume of PBS and stored on ice for 1-5 hours. The measurements of efflux were performed with FluoroMax-3 fluorometer system with exication wavelength set to 480 nm, and emission wavelength set to 560 nm. The R6G efflux was initiated by addition of 0.1% glucose, cell density in fluorometric cuvette was 10⁶ cells/ml.

Survival Assay.

Exponentially grown cells were taken and treated with indicated amounts of C₁₂TPP or SKQ1 for 3 hours. Then cell suspensions were plated on solid YPD medium and incubated for 48 hours, the number of formed colonies were counted. 100% refers to the number of colony forming units (CFU) in yeast suspension at the beginning of experiment.

Statistics

Wilcoxon signed-ranked tests (n=3-11) and the slopes of the fluorometric curves were calculated using R software package. Error bars on figures represent standard errors of the mean.

Results and Discussion

To see whether C₁₂TPP can inhibit MDR pump activity we first decided to find the particular pump which extrudes it from S. cerevisiae cells. We performed genetic screening using yeast S. cerevisiae with a yeast genome library on a multicopy plasmid. As a result it was found that a plasmid with chromosome II fragment (coordinates from 216287 to 222344) harboring two genes, LDB7 and PDR3, provided a significant increase of resistance to 20 μM C₁₂TPP. Pdr3p is a transcription factor responsible for upregulation of a set of ABC-transporters including three unspecific MDR pump genes: PDR5, SNQ2 and YOR1 [7-9]. These data strongly suggested that C₁₂TPP is a substrate of one of these pumps. To find a specific pump responsible for C₁₂TPP detoxication we produced a set of mutant strains with corresponding genes under the control of galactose inducible, glucose repressible GAL promoters.

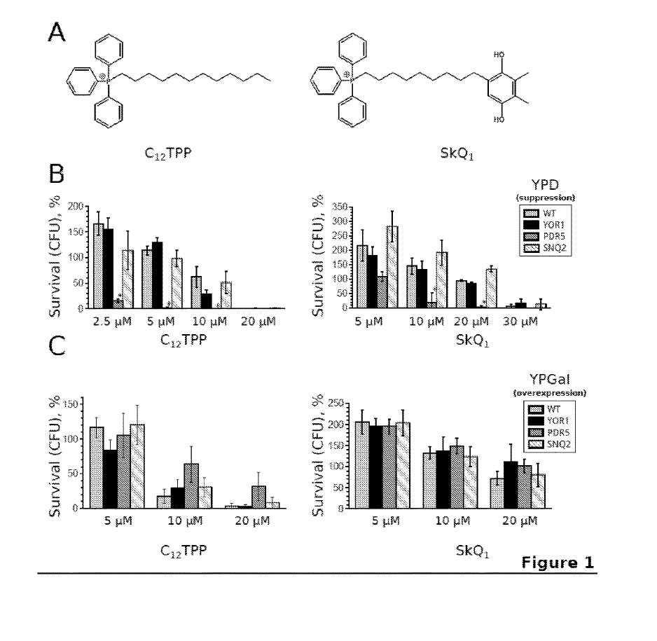

It appeared that expression of PDR5 is critical for resistance of cells to C₁₂TPP, whereas repression of SNQ2 or YOR1 did not show a statistically significant effect (FIG. 1B). Accordingly, overexpression of PDR5 provided a moderate protection to high concentrations of C₁₂TPP (FIG. 1C). We did not observe any effect of MDR overexpression in the case of SkQ1. Possibly, basal levels of expression of the pumps that extrude SkQ₁ are sufficient to prevent its toxicity.

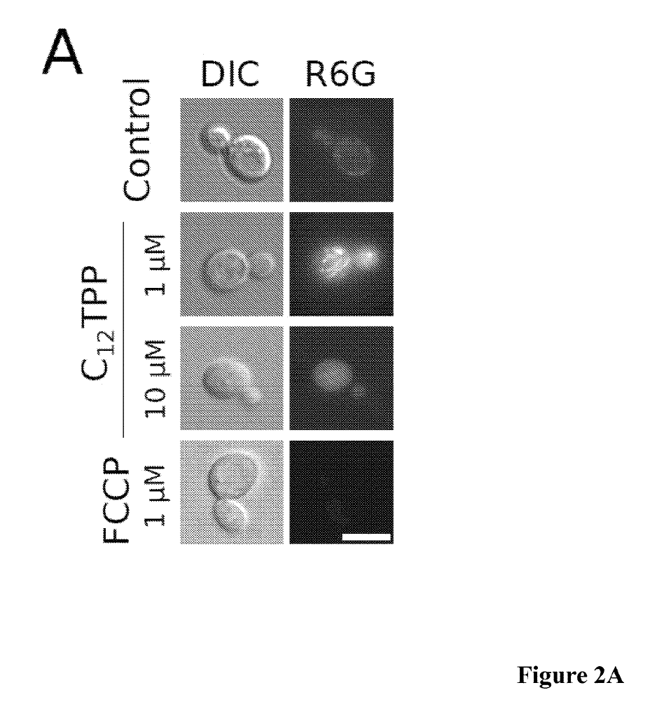

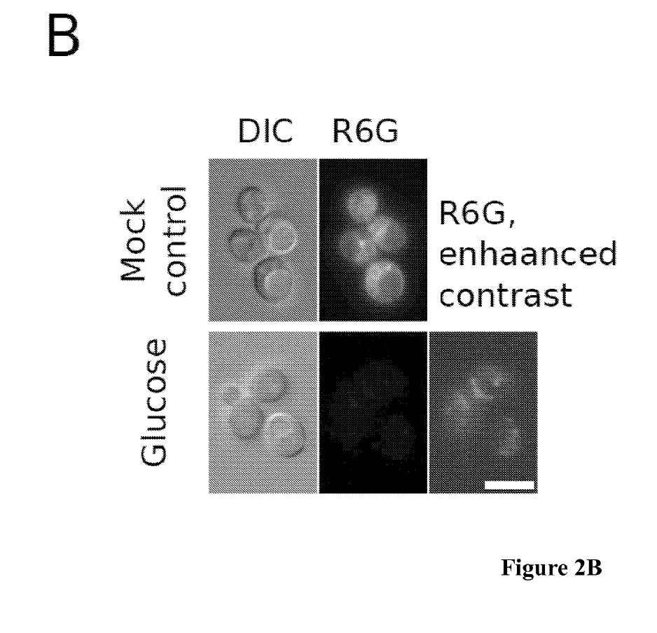

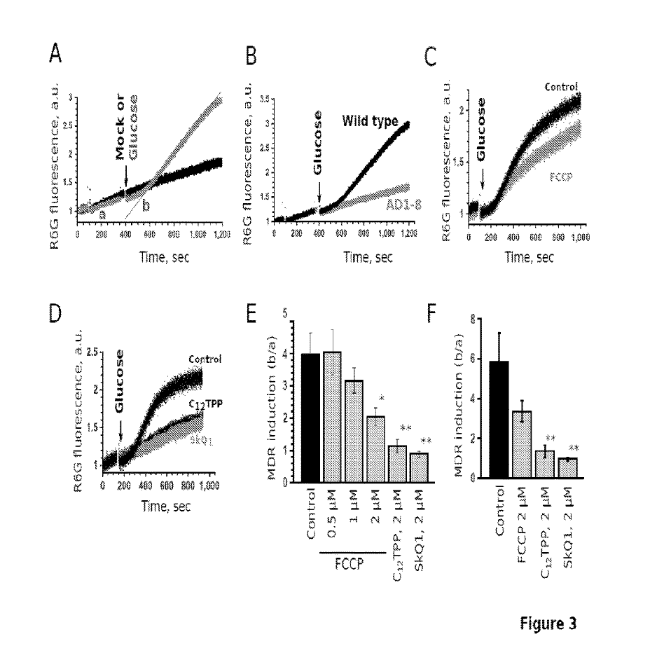

One possible explanation for these results is that C₁₂TPP is extruded by Pdr5 from yeast cells, and that C₁₂TPP competes with other PDR5 substrates. Indeed, low concentrations of C₁₂TPP enhance the staining of yeast cells with positively charged fluorescent dye rhodamine 6G (FIG. 2A) which is likely to be a Pdr5p substrate [6]. Accordingly, in energy deprived cells R6G accumulates in high concentration, and after the addition of glucose the fluorescence inside cells is decreased and remains mostly in polarized mitochondria (FIG. 2B). We measured the efflux of R6G from intact yeast cells using fluorometric method. The efflux was visualized by fluorescent spectroscopy: rhodamine 6G is self-quenched in cells and therefore the R6G release results in a detectable increase of total fluorescence of the dye (FIG. 3A). Importantly, R6G release is completely abrogated in MDR-negative cells lacking all major PDR genes (FIG. 3B).

The addition of uncoupler FCCP was able to partially prevent R6G efflux (FIG. 3C), while C₁₂TPP and SkQ1 appeared to be much more efficient in this respect (FIGS. 3D, E). Apparently, this effect of lipophilic cations could be either due to a direct inhibition of MDR pumps, or a result of mitochondrial uncoupling causing a decrease of ATP concentration. However, the latter is seemed unlikely because FCCP is a much more potent uncoupler than C₁₂TPP [10]. Moreover, this possible effect of C₁₂TPP can be ruled out because it was also able to inhibit R6G efflux in cells pretreated with an excess of NaN₃ (FIG. 3F). NaN₃ inhibits both respiratory chain [11] and mitochondrial ATP synthase [12]. At the same time, in contrast to another ATP synthase inhibitor, oligomycin, it is not as efficient in inhibiting MDR activity in yeast [6]. Therefore, in the presence of 10 mM NaN₃, the contribution of mitochondria in ATP supply appears to be minimal and the effects of C₁₂TPP and SkQ1 i are mainly due to the repression of MDR.

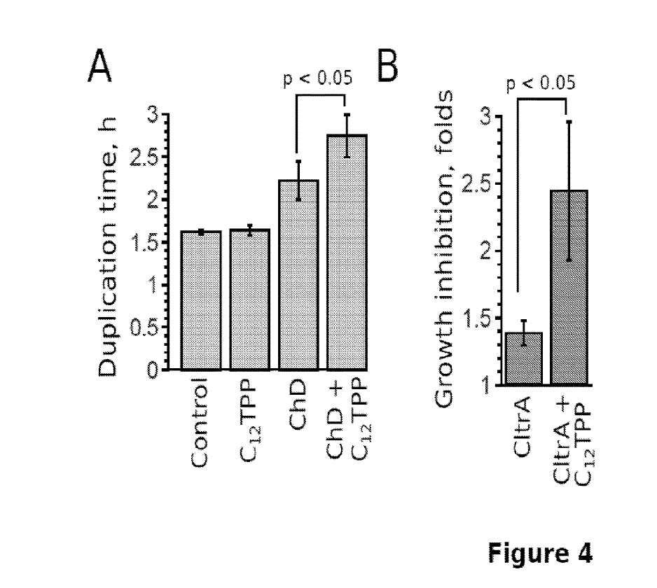

Is it possible to induce uptake of other PDR5 substrates by addition of C₁₂TPP? To test this we measured the growth rate of yeast cells in the presence of protein synthesis inhibitor cycloheximide D (ChD) which is a well known substrate of Pdr5 [13]. It was found that 1 μM of C₁₂TPP significantly enhances the inhibitory effect of ChD (FIG. 4A). Moreover, C₁₂TPP augments the action of antifungal clotrimazole (FIG. 4B), which was also previously reported to be a substrate of yeast MDR-pumps [14]. Importantly, we did not detect any significant stimulation of the toxicity for another antifungal—amphotorecine B, which acts in the plasma membrane and is not attributed to ABC pumps substrates.

To conclude, together with the previous observations our data show that C₁₂TPP inhibits the multidrug resistance and suggest that this inhibition is due to a futile cycle of its extrusion followed by returning back into the cells. Therefore it can be used to increase cellular uptake of other ABC-transporter substrates (including other amphiphilic compounds). Remarkably, earlier it was shown that C₁₂TPP facilitates the transport of anionic molecules across the membranes: fluorescent dye fluoresceine [15], fatty acids [16] and anionic uncouplers [10]. Thus, C₁₂TPP appears to be a universal plasma membrane permeabiliser. Therefore, our findings make it a promising supplement for anti-mycotic drugs to prevent their efflux from cells.

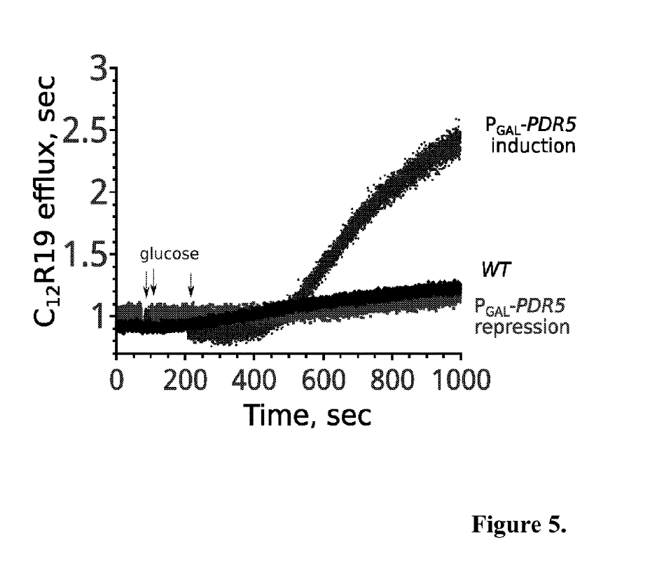

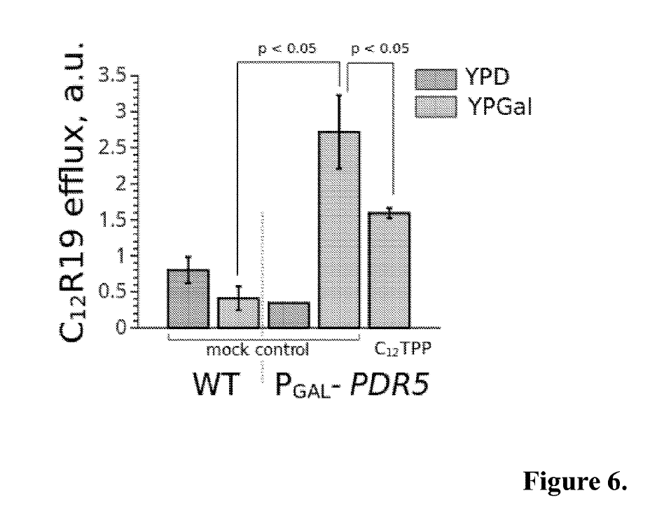

In the next experiment we have tested dodecylated rhodamine C₁₂R1 (see Antonenko et al. 2012; doi: 10.1074/jbc.M110.212837). We found that this compound accumulated in energy deprived yeast cells in a similar way to rhodamine 6G. The addition of glucose to such cells promoted the efflux of C₁₂R1. The efflux was detected by decrease of self quenching in fluorometer cuvette by increase of fluorescent signal (excitation wavelength was 480 nm, emmision wavelength 560 nm). We found that efficient efflux could be observed only in cells overexpressing PDR5 genes (FIGS. 5, 6). This result indicates that, while being exported by Pdr5p multidrug efflux pump, C₁₂R1 could be withdrawn from cell only under condition of PDR5 overexpression. Next, we have shown that C₁₂TPP prevents efflux of C₁₂R1. This confirms that C₁₂TPP and C₁₂R1 are substrates of MDR pump Pdr5p.

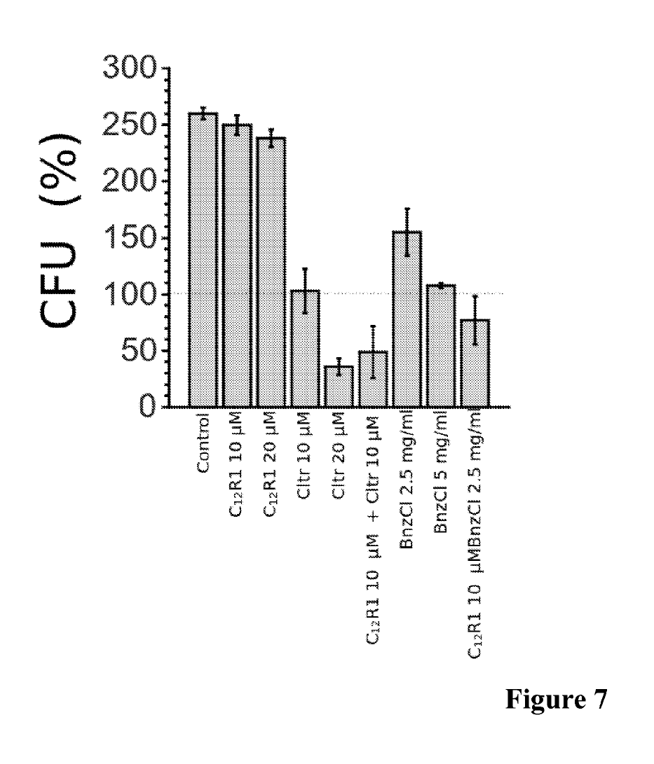

Next we tested the ability of C₁₂R1 to increase sensitivity of fungi cell to common antifungals. It was found that in non-toxic concentrations C₁₂R1 increase the toxicity of antifungal clotrimazole and benzalkonium chloride (FIG. 7).

### Experimental Example 2

### Anti-Bacterial Effect of Compounds of General Formula 1

Multidrug efflux transporters cause serious problems in the treatment of bacterial infections and cancer chemotherapy. In gram-negative bacteria, such as E. coli, transporters belonging to the resistance-nodulation-cell division (RND) family are particularly effective in generating resistance because they form a tripartite complex with periplasmic proteins and an outer membrane protein channel [Du D. et al., (2014) Structure of the AcrAB-To1C multidrug efflux pump. Nature, 509, 512-515]. The AcrAB-To1C efflux pump is able to transport great number of compounds with little chemical similarity, thus conferring resistance to a broad spectrum of antibiotics [Pos K. M. (2009) Drug transport mechanism of the AcrB efflux pump. Biochem. Biophys. Acta, 1794, 782-793; Seeger M. A. et al., (2008) The AcrB Efflux Pump: Conformational Cycling and Peristalsis Lead to Multidrug Resistance. Current Drug Targets, 2008, 9, 729-749; Sulavik M. C. et al., (2001) Antibiotic Susceptibility Profiles of Escherichia coli StrainsLacking Multidrug Efflux Pump Genes. Antimicrob. Agents Chemother., 45, 4, 1126-1136]. TheAcrAB-To1C system is composed of the RND transporter AcrB, membrane fusion protein AcrA, and multifunctional outer membrane channel To1C. To1C is the universal outer membrane channel for export of toxins and drug efflux [Andersen C., Hughes C., Koronakis V. (2001) Protein export and drug efflux through bacterial channel-tunnels. Curr Opin Cell Biol., 13, 412-416].

To1C interacts with a variety of inner membrane transporters and enables E. coli to expel structurally diverse molecules out of the cell. In E. coli, AcrB, AcrD, AcrEF, MdtABC, and MdtEF belong to the RND transporters and require To1C to function. [Horiyama T. and Nishino K. (2014) AcrB, AcrD, and MdtABC Multidrug Efflux Systems Are Involved in Enterobactin Export in Escherichia coli. PLoS ONE 9, 9, e10864].

### Antibacterial Activity of 1 μM SkQ1.

Growing in the liquid medium method was selected as the method to test antibacterial activity of SkQ1. Overnight bacterial cells cultures were diluted in fresh LB media. Bacterial cell culture (5×10⁶ cells/ml) were inoculated 200 μl into 96-well plates (Eppendorf, Eppendorf AG, Hamburg, Germany) and SkQ1 or CₙTPP were added to reach various final concentrations in the range from 0.5 to 200 μM. Cells were left to grow for 21 hours at 37° C. Optical density at 600 nm was measured using an AnthosZenyth 3100 multimode reader (Anthos Labtec, Austria) during incubation.

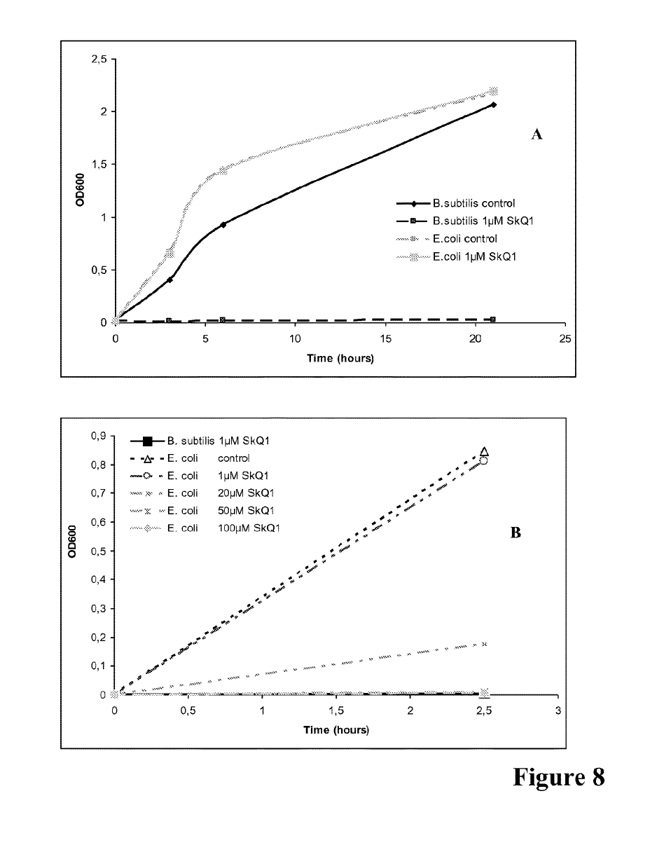

We selected Ecsherishia coli cells as a Gram-negative model organism and Bacillus subtilis cells as a Gram-positive model organism. SkQ1 was selected as a model compound of formula 1 with antioxidant moiety. To select a proper concentration of a compound of formula 1 we performed growth inhibition assays for several SkQ1 concentrations (see FIGS. 8 A and B). It can be concluded from the results of this experiment that 1 μM SkQ1 is the minimal inhibitory concentration (MIC) for B. subtilis cells. But this concentration did not inhibit significantly growth of E. coli cells. 50 μM SkQ1 was found to be the MIC for E. coli cells. These results indicate that SkQ1 has strong antimicrobial activity against gram-positive bacteria cells and mild antimicrobial activity against gram-negative bacteria.

### Antibacterial Activity of CₙTPP.

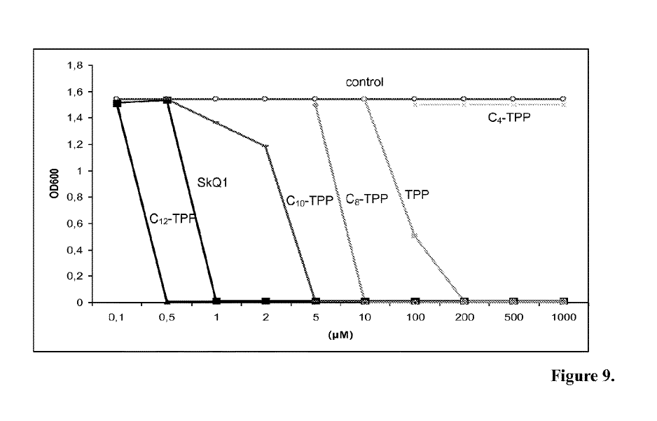

We tested resistance of B. subtilis to triphenylphosphonium derivatives from selected ranges of concentration, such as, dodecyltriphenylphosphonium (C₁₂TPP), decyltriphenylphosphonium (C₁₀TPP), octyltriphenylphosphonium (C₈TPP), and butyltriphenylphosphonium (C₄TPP). We performed growth inhibition assays for several triphenylphosphonium derivative concentrations (see FIG. 9) and found the MIC for B. subtilis for all used triphenylphosphonium derivatives, except C₄TPP. A gradual increase of the inhibiting concentration for Cₙ-TPP with growing length of the hydrocarbonic tail was observed. Unexpectedly. MIC of C₄TPP could not be reached in this experiment. These results indicate that CₙTPPs, except C₄TPP, have antimicrobial activity against gram-positive bacterial cells.

### Antibacterial Activity of Other Compounds of General Formula 1.

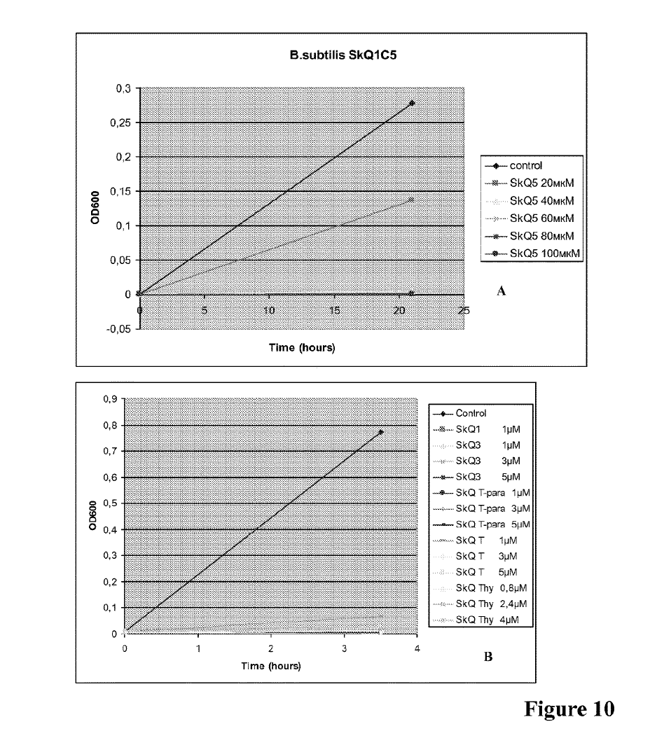

In the following experiments we used different compounds of general formula 1 such as SkQ5, SkQT, SkQThy, SkQT-para, and SkQ3. We performed growth inhibition assays for several concentrations of the selected compounds (see FIGS. 10A and B) and found that all of the selected triphenylphosphonium derivatives have antimicrobial activity. These results indicate that all selected compounds of general formula 1 have antimicrobial activity based on the length of the hydrocarbon linker and antioxidant structure (i.e. variables A, in and n of the general formula 1).

Antibacterial Activity of SkQ1 against To1C-Required RND Efflux Pumps (MDR).

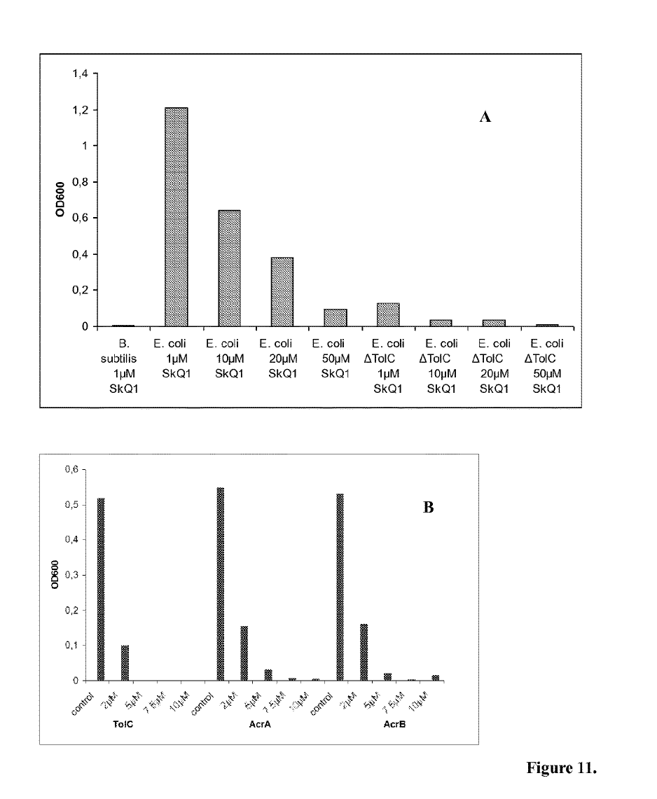

In this experiment we have tested whether To1C-dependent transporter is responsible for resistance of E. coli's cells to SkQ1. In the experiment we used single-gene knockout mutants from Keio collection [Baba T., et al., (2006)]. Construction of Escherichia coli K-12 in-frame, single-gene knockout mutants: the Keio collection. Mol. Syst. Biol. 2, 2006.0008.]. We found that To1C-deletion mutant has lost resistance to SkQ1, and demonstrated same sensitivity as B. subtilis, with minimal bactericidal concentration around 2 μM. Similar results were observed for AcrA and AcrB deletion mutants (see FIG. 11). We used other knockout mutants of genes encoding proteins of transporters that require To1C for their function. Deletion of all of transporter genes, except AcrA and AcrB, did not affect resistance of E. coli cells (data not shown). In contrast, deletion of genes acrA or acrB dramatically changed the sensitivity of E. coli's cells to SkQ1. AcrA- or AcrB-deletion mutants showed similar sensitivity to that of the To1C-deletion mutant (Table 1). These results show that resistance of E. coli to SkQ1 can be a result of AcrAB-To1C transporters activity.

### Antibacterial Activity of C_{C}TPP Against To1C-Required RND Efflux Pumps.

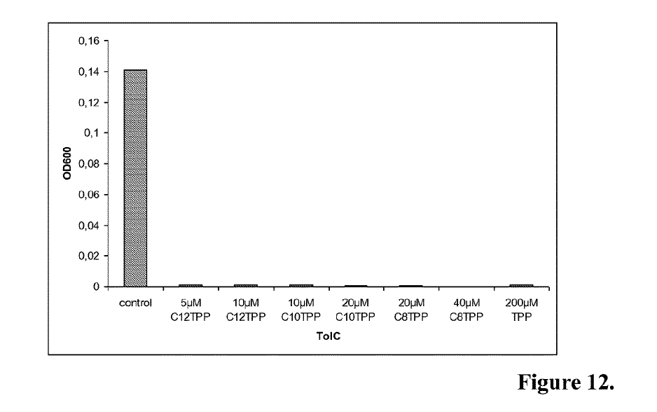

We tested resistance of the To1C-deletion mutant to other compounds of general formula 1, such as, dodecyltriphenylphosphonium (C₁₂TPP), decyltriphenylphosphonium (C₁₀TPP), octyltriphenylphosphonium (C₈TPP), butiltriphenylphosphonium (C₄TPP). Concentrations of the compounds were selected on the basis of an assumption that the minimal concentration would be about the minimal bactericidal concentration of the compounds for B. subtilis (FIG. 12), and maximal concentration did not exceed half of minimal bactericidal concentration of the compounds for E. coli cells (data not shown). We observed that To1C-deletion mutant also lost resistance to compounds of general formula 1 (FIG. 12). To1C-deletion mutant had similar sensitivity to CₙTPP as B. subtilis. These results indicates that all selected CₙTPPs have antimicrobial activity against To1C-deletion mutant, showing that AcrAB-To1C transporter is responsible for CₙTPP efflux from the cell.

| | IMPOSSIBILITY OF GROWTH | | | |
|---|---|---|---|---|
| | 5 μM SkQ1 | 30 μM SkQ1 | 5 μM C12-TPP | 30 μM C12-TPP |
| E. coli | NO | NO | NO | NO |
| ΔTolC | YES | YES | YES | YES |
| ΔAcrA | YES | YES | YES | YES |
| ΔAcrB | YES | YES | YES | YES |
| ΔAcrD | NO | NO | NO | NO |
| ΔAcrE | NO | NO | NO | NO |
| ΔAcrF | NO | NO | NO | NO |
| ΔMdtA | NO | NO | NO | NO |
| ΔMdtB | NO | NO | NO | NO |
| ΔMdtC | NO | NO | NO | NO |
| ΔMdtE | NO | NO | NO | NO |
| ΔMdtF | NO | NO | NO | NO |

Table 1. Results of growth inhibition assay for deletion mutants of genes encoding To1C-required efflux pumps proteins. Deletion strains JW5503 (devoid of the to1C gene), JW0452 (devoid of the acrA gene), JW0451 (devoid of the acrB gene), JW2454 (devoid of the acrD gene), JW3233 (devoid of the acrE gene), JW3234 (devoid of the acrF gene), JW5338 (devoid of the mdtA gene), JW2060 (devoid of the mdtB gene), JW2061 (devoid of the mdtC gene), JW3481 (devoid of the mdtE gene), and JW3482 (devoid of the mdtF gene) have been described in Baba T., et al., (2006). Construction of Escherichia coli K-12 in-frame, single-gene knockout mutants: the Keio collection. Mol. Syst. Biol. 2, 2006.0008.

### Ethidium Bromide Accumulation Experiment.

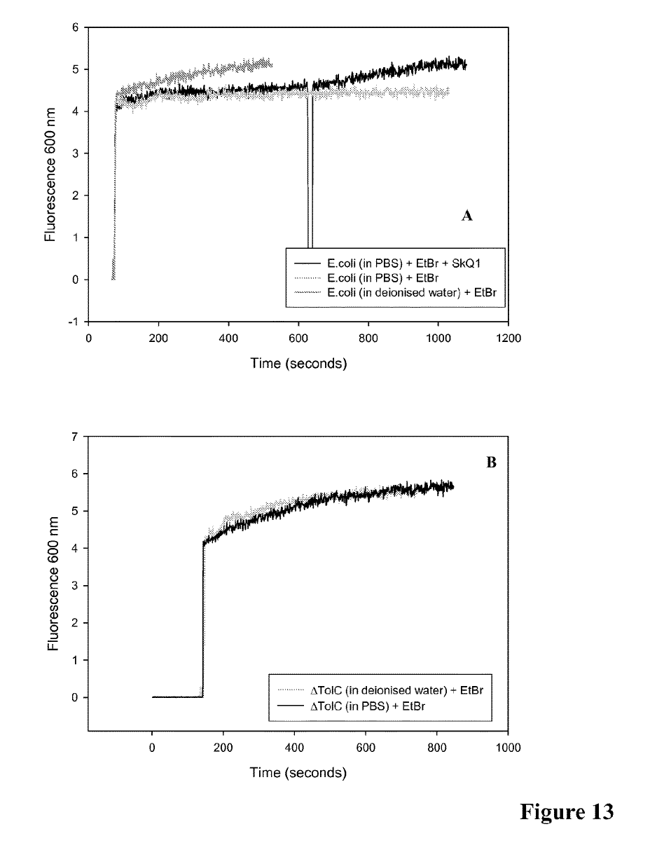

To confirm whether AcrAB-To1C is the appropriate transporter, we used a test based on the measurement of ethidium bromide leakage through intact bacterial membranes. If SkQ1 is a substrate for AcrAB-To1C transporter, then it is expected that ethidium bromide leakage through bacterial membranes will be comparable to ethidium bromide leakage during osmotic shock when SkQ1 concentration is about minimal bactericidal concentration for E. coli. Bacterial cells were prepared and resuspended in PBS. For emulation of cell membrane leakage bacterial cells were resuspended in deionized water (Milli-Q water purification system, EMD Millipore, Billerica, Mass.). Ethidium bromide was added (20 μg/ml) and the change of fluorescence intensity was recorded on Fluorat-02-Panorama (Lumex Instruments, Russia) spectrofluorometer. For increasing the accumulation of ethidium bromide, SkQ1 was added to reach a concentration of 50 μM. Usually, wild type E. coli cells have resistance to ethidium bromide up to 800 μg/ml, therefore using concentration of ethidium bromide 20 μg/ml, we did not expect to see ethidium bromide leakage through bacterial membranes except in case of adding exhausting concentration of substrate. We observed ethidium bromide accumulation when ethidium bromide concentration was 40-times less than MIC for E. coli cells that only can be the result of collateral action of SkQ1 and ethidium bromide as substrates for AcrAB-To1C transporter. As a result it was confirmed that SkQ1 is a substrate for AcrAB-To1C transporter, because we observed ethidium bromide leakage through bacterial membranes, comparable to ethidium bromide leakage during osmotic shock, when we added SkQ1 to reach minimal bactericidal concentration for E. coli (FIGS. 13A and B).

### REFERENCES FOR EXPERIMENTAL EXAMPLE 1

[1] S. Shukla, V. Yadav, G. Mukhopadhyay, R. Prasad, Ncb2 is involved in activated transcription of CDR1 in azole-resistant clinical isolates of Candida albicans., Eukaryot. Cell. 10 (2011) 1357-66. doi:10.1128/EC.05041-11.
[2] B. A. McManus, E. McGovern, G. P. Moran, C. M. Healy, J. Nunn, P. Fleming, et al., Microbiological screening of Irish patients with autoimmune polyendocrinopathy-candidiasis-ectodermal dystrophy reveals persistence of Candida albicans strains, gradual reduction in susceptibility to azoles, and incidences of clinical signs of oral candid, J. Clin. Microbiol. 49 (2011) 1879-89. doi:10.1128/JCM.00026-11.
[3] Y. N. Antonenko, A. V Avetisyan, L. E. Bakeeva, B. V Chernyak, V. A. Chertkov, L. V Domnina, et al., Mitochondria-targeted plastoquinone derivatives as tools to interrupt execution of the aging program. 1. Cationic plastoquinone derivatives: synthesis and in vitro studies., Biochem. Biokhimii□a. 73 (2008) 1273-87.
[4] A. Decottignies, A. M. Grant, J. W. Nichols, H. de Wet, D. B. McIntosh, A. Goffeau, ATPase and multidrug transport activities of the overexpressed yeast ABC protein Yor1p., J. Biol. Chem. 273 (1998) 12612-22.
[5] F. Sherman, Getting started with yeast., Methods Enzymol. 350 (2002) 3-41.
[6] M. Kolaczkowski, M. van der Rest, A. Cybularz-Kolaczkowska, J. P. Soumillion, W. N. Konings, A. Goffeau, Anticancer drugs, ionophoric peptides, and steroids as substrates of the yeast multidrug transporter Pdr5p., J. Biol. Chem. 271 (1996) 31543-8.
[7] D. J. Katzmann, T. C. Hallstrom, M. Voet, W. Wysock, J. Golin, G. Volckaert, et al., Expression of an ATP-binding cassette transporter-encoding gene (YOR1) is required for oligomycin resistance in Saccharomyces cerevisiae., Mol. Cell. Biol. 15 (1995) 6875-83.
[8] D. J. Katzmann, P. E. Burnett, J. Golin, Y. Mahé, W. S. Moye-Rowley, Transcriptional control of the yeast PDR5 gene by the PDR3 gene product., Mol. Cell. Biol. 14 (1994) 4653-61.
[9] A. Decottignies, L. Lambert, P. Catty, H. Degand, E. A. Epping, W. S. Moye-Rowley, et al., Identification and characterization of SNQ2, a new multidrug ATP binding cassette transporter of the yeast plasma membrane., J. Biol. Chem. 270 (1995) 18150-7.
[10] Y. N. Antonenko, L. S. Khailova, D. A. Knorre, O. V Markova, T. I. Rokitskaya, T. M. Ilyasova, et al., Penetrating cations enhance uncoupling activity of anionic protonophores in mitochondria., PLoS One. 8 (2013) e61902. doi:10.1371/journal.pone.0061902.

[11] W. O. James, The Use of Respiratory Inhibitors, Annu. Rev. Plant Physiol. 4 (1953) 59-90. doi:10.1146/annurev.pp.04.060153.000423.

[12] P. Mitchell, J. Moyle, Activation and inhibition of mitochondrial adenosine triphosphatase by various anions and other agents., J. Bioenerg. 2 (1971) 1-11.

[13] G. Leppert, R. McDevitt, S. C. Falco, T. K. Van Dyk, M. B. Ficke, J. Golin, Cloning by gene amplification of two loci conferring multiple drug resistance in Saccharomyces., Genetics. 125 (1990) 13-20.

[14] J. Golin, S. V Ambudkar, M. M. Gottesman, A. D. Habib, J. Sczepanski, W. Ziccardi, et al., Studies with novel Pdr5p substrates demonstrate a strong size dependence for xenobiotic efflux., J. Biol. Chem. 278 (2003) 5963-9. doi:10.1074/jbc.M210908200.

[15] T. I. Rokitskaya, N. V Sumbatyan, V. N. Tashlitsky, G. A. Korshunova, Y. N. Antonenko, V. P. Skulachev, Mitochondria-targeted penetrating cations as carriers of hydrophobic anions through lipid membranes., Biochim. Biophys. Acta. 1798 (2010) 1698-706. doi:10.1016/j.bbamem.2010.05.018.

[16] F. F. Severin, I. I. Severina, Y. N. Antonenko, T. I. Rokitskaya, D. A. Cherepanov, E. N. Mokhova, et al., Penetrating cation/fatty acid anion pair as a mitochondria-targeted protonophore., Proc. Natl. Acad. Sci. U.S.A. 107 (2010) 663-8. doi:10.1073/pnas.0910216107.

What is claimed is:

1. A method for treating a microbial infection in an infected subject comprising administering to the subject an antimicrobial composition comprising a therapeutically effective amount of one or more lipophilic cation of structural formula 1: 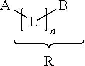
wherein A is hydrogen atom (H) or an antioxidant moiety having the following structure: 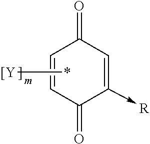
and/or reduced (quinole) form thereof,
wherein m is an integer from 0 to 3;
each Y is methyl;
L is a linker group which is a straight or branched hydrocarbon chain which can be optionally substituted by one or more substituents and optionally contains one or more double or triple bonds; and
B is a lipophilic cation,
and a pharmacologically acceptable anion.

2. The method according to claim 1 wherein the one or more lipophilic cation is one or more of SkQ1, SkQ3, SkQ4, SkQ5, SkQR1, SkQRB1, SkQB1, SkQBP1, SkQT, SkQThy, SkQB, CₙTPP (wherein n is from 5 to 12), CₙR1 (wherein n is from 4 to 12), CₙBerb (wherein n is from 4 to 12), and CₙPalm (wherein n is from 4 to 12).

3. The method according to claim 1 or 2, wherein the microbial infection is caused by bacteria.

4. The method according to claim 3 wherein the microbial infection is caused by Gram-negative bacteria.

5. The method according to claim 3 wherein the disease is caused by Gram-positive bacteria.

6. The method according to claim 3 wherein the disease is caused by Gram-negative bacteria and Gram-positive bacteria.

7. The method according to claim 1 or 2, wherein the disease is caused by fungi.

8. The method according to claim 1 or 2, wherein the disease is caused by bacteria and fungi.

9. The method according to claim 1, 2, 3, 4, 5, 6, 7, or 8, further comprising administering to the subject another one or more antimicrobial compound or one or more antimicrobial agent.

10. The method according to claim 9, wherein the additional one or more antimicrobial compound is one or more antibacterial antibiotic.

11. The method according to claim 9, wherein the one or more additional antimicrobial compound is one or more antifungal compound.

12. The method according to claim 9, wherein the additional one or more antimicrobial agent is one or more antiseptic.

13. The method according to claim 9, wherein the additional one or more antimicrobial compound is one or more MDR pump inhibitor.

14. The method according to claim 9, wherein the additional one or more antimicrobial compound is one or more MDR pump substrate.

15. A pharmaceutical composition with antimicrobial properties comprising a therapeutically effective amount of one or more lipophilic cation of structural formula 1: 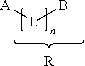
wherein A is hydrogen atom (H) or an antioxidant moiety having the following structure: 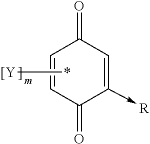
and/or reduced (quinole) form thereof
wherein m is an integer from 0 to 3;
each Y is methyl;
L is a linker group which is straight or branched hydrocarbon chain which can be optionally substituted by one or more substituents and optionally contains one or more double or triple bonds; and
B is a lipophilic cation,
and a pharmacologically acceptable anion,
and one or more additional antimicrobial compound or one or more antimicrobial agent.

16. The pharmaceutical composition according to claim 15 wherein the one or more lipophilic cation is one or more of SkQ1, SkQ3, SkQ4, SkQ5, SkQR1, SkQRB1, SkQB1, SkQBP1, SkQT, SkQThy, SkQB, CₙTPP (wherein n is from 5 to 12), CₙR1 (wherein n is from 4 to 12), CₙBerb (wherein n is from 4 to 12), and CₙPalm (wherein n is from 4 to 12).

17. The pharmaceutical composition according to claim 15 or 16, wherein the additional one or more antimicrobial compound is one or more antibacterial antibiotic.

18. The pharmaceutical composition according to claim 15 or 16, wherein the additional one or more antimicrobial substance is one or more antifungal compound.

19. The pharmaceutical composition according to claim 15 or 16, wherein the additional one or more antimicrobial agent is one or more antiseptic.

20. The pharmaceutical composition according to claim 15 or 16, wherein the additional one or more antimicrobial substance is one or more MDR pump inhibitor.

21. The pharmaceutical composition according to claim 15 or 16, wherein the additional one or more antimicrobial compound is one or more MDR pump substrate.
